(19) United States
(12) Patent Application Publication
(10) Pub. No.: US 2009/0093420 A1
(43) Pub. Date: Apr. 9, 2009

(54) PROCESSES FOR THE PREPARATION OF AZITHROMYCIN

(75) Inventors: Manish Purohit, Madhya Pradesh (IN); Siddiqui Mohammad Jaweed Mukarram, Maharashtra (IN); Pankaj Kumar Naithani, Uttranchal (IN); Rai Kumar Pokalwar, Maharashtra (IN); Prakash Maganlal Sutariya, Gujarat (IN)

Correspondence Address: SCHWEGMAN, LUNDBERG & WOESSNER, P.A., P.O. BOX 2938, MINNEAPOLIS, MN, 55402, US

(73) Assignee: WOCKHARDT LIMITED, MUMBAI (IN)

(21) Appl. No.: 12/096,991

(22) PCT Filed: Jan. 12, 2007
(86) PCT No.: PCT/IB07/00085

§ 371 Date: Sep. 30, 2008

(30) Foreign Application Priority Data
Jan. 12, 2006 (IN) 43/MUM/06
Aug. 31, 2006 (IN) 1376/MUM/06
Aug. 31, 2006 (IN) 1377/MUM/06
Aug. 31, 2006 (IN) 1381MUM/06

(51) Int. Cl. A61K 31/7048; C07H 17/08; A61P 31/04

(52) U.S. Cl. 514/29; 536/7.2

(57) ABSTRACT

The invention relates to processes for the preparation of anhydrous azithromycin. The invention also relates to a one-pot process for the preparation of azithromycin without isolation of intermediates. The invention also relates to pharmaceutical compositions that include the anhydrous azithromycin or a pharmaceutically acceptable salt thereof.

### FIELD OF THE INVENTION

The field of the invention relates to processes for the preparation of anhydrous azithromycin. The invention also relates to a one-pot process for the preparation of azithromycin without isolation of intermediates. The invention also relates to pharmaceutical compositions that include the anhydrous azithromycin or a pharmaceutically acceptable salt thereof.

### BACKGROUND OF THE INVENTION

Chemically, azithromycin is (2R,3S,4R,5R,5R,10R,11R,12S,13S,14R)-13-[(2,6-dideoxy-3-C-methyl-3-O-methyl-a-L-ribo-hexopyranosyl)oxy]-2-ethyl-3,4,10-trihydroxy-3,5,6,8,10,12,14-heptamethyl-11-[[3,4,6-trideoxy-3-(dimethylamino)-b-D-xylo-hexopyranosyl]oxy]-1-oxa-6-azacyclopentadecan-15-one having the structural Formula I. Azithromycin is indicated for the treatment of patients with mild to moderate infections caused by susceptible strains of the microorganisms. It is also indicated for the treatment of acute bacterial sinusitis and community-acquired pneumonia.

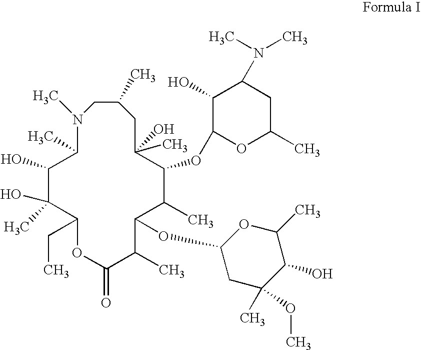

Several processes have been reported for the preparation of azithromycin for example, in U.S. Pat. Nos. 4,474,768; 4,517,359; 6,420,537; and CA 1191843.

U.S. Pat. No. 4,328,334 discloses a method of preparing the compound of formula (III) by reducing the compound of formula (II) in a methanol at lower temperature with sodium borohydride.

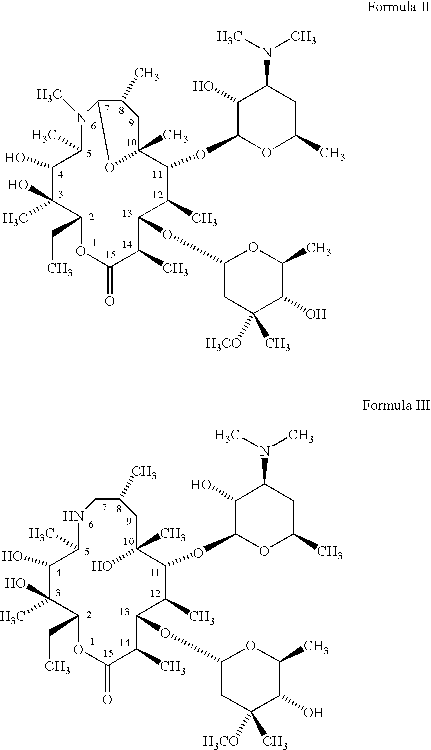

Azithromycin has been reported to exist in several hydrated forms. U.S. Pat. No. 6,268,489 discloses a stable dihydrate form of azithromycin. U.S. Pat. No. 6,855,813 discloses stable form of azithromycin monohydrate. U.S. Pat. No. 6,977,243 discloses crystal forms of azithromycin and azitbromycin as a sesquihydrate.

EP 1313749 B1 discloses a method for the preparation of anhydrous azithromycin by removing an organic solvent from the solution containing the hydrated compound in an organic solvent or a solution of the hydrated compound in a mixture of an organic solvent and water.

U.S. Pat. No. 4,963,531 discloses a process for the preparation of azithromycin as a dihydrate. The patent also discloses that on storage at low humidity the azithromycin dihydrate loses water and samples of azithromycin mono- and di-hydrate stored at higher humidity rapidly absorbed water. Therefore, the water percentage (percent hydration) in the crystals can vary depending on the relative humidity during storage.

### SUMMARY OF THE INVENTION

In one general aspect there is provided a process for the preparation of azithromycin. The process includes:
a) reducing 6,9 imino ether compound of Formula II

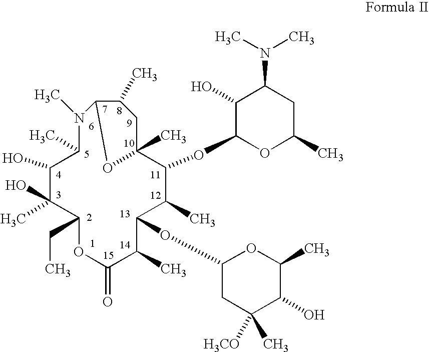

with a boron containing reducing agent, to get a compound of Formula III or its boron complex;

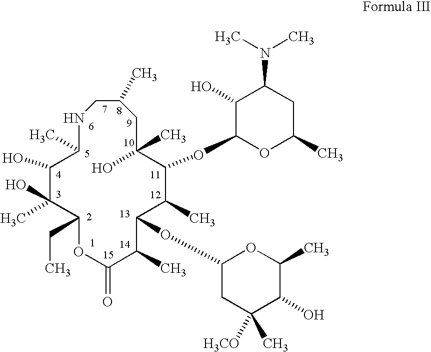

b) optionally, breaking the boron complex to get the compound of Formula III; and
c) treating the compound of Formula III with formic acid and formaldehyde to get the azithromycin.

In another general aspect there is provided a process for the preparation of azithromycin. The process includes:
a) reducing 6,9 imino ether compound of Formula II

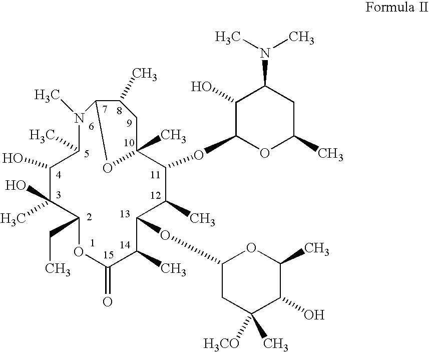

with a boron containing reducing agent, to get a boron complex of compound of Formula III;

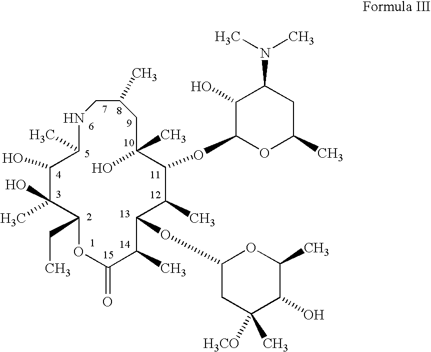

b) extracting the boron complex in one or more organic solvents;
c) treating the organic solvent containing the boron complex with malic acid;
d) N-methylating with formic acid and formaldehyde; and
e) isolating the azithromycin.

In another general aspect there is provided a process for the preparation of azithromycin. The process includes:
a) reducing 6,9 imino ether compound of Formula II

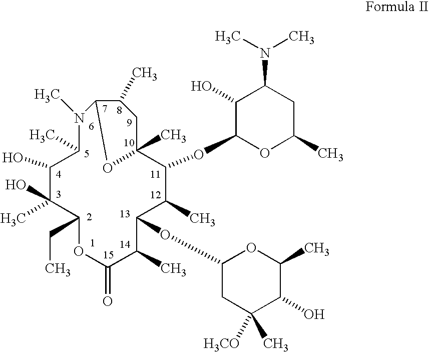

with a boron containing reducing agent, to get a boron complex of compound of Formula III;

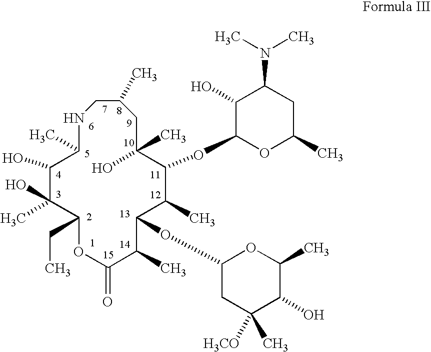

b) extracting the boron complex in one or more organic solvents;
c) N-methylating with formic acid and formaldehyde to get azithromycin boron complex;
d) isolating the azithromycin boron complex from reaction mass; and
e) breaking the boron complex of azitbromycin to get the azithromycin.

Embodiments of the invention include one or more of the following features. For example, the process is carried out in one pot and no intermediate is isolated.

In another aspect there is provided a storage stable anhydrous form of azithromycin.

In another general aspect there is provided a process for the preparation of stable form of anhydrous azithromycin. The process includes removing the moisture from hydrated forms of azithromycin; adding one or more water miscible solvents; and isolating the anhydrous form of azithromycin by the removal of the solvents.

The anhydrous form of azithromycin may have, for example, a purity of 99% or more when measured by HPLC and a moisture content of about 1.0% w/w or less.

Removing the solvents may include, for example, one or more of filtration, filtration under vacuum, distillation, distillation under vacuum, evaporation, decantation and centrifugation. The process may include further forming of the product so obtained into a finished dosage form.

The process may include further drying of the product obtained.

In another general aspect there is provided a pharmaceutical composition that includes a therapeutically effective amount of the stable form of anhydrous azithromycin or a salt thereof; and one or more pharmaceutically acceptable carriers, excipients or diluents.

The details of one or more embodiments of the inventions are set forth in the description below. Other features, objects and advantages of the inventions will be apparent from the description and claims.

### DETAILED DESCRIPTION OF THE INVENTION

The inventors have developed a process for the preparation of azithromycin using 6,9 imino ether compound of formula II as the starting material. More particularly, the inventors have developed a process with reduced reaction time cycle for converting 6,9-Imino ether compound of Formula II to cyclic amine compound of Formula III. The boron complex of the compound of Formula III obtained after reducing the compound of Formula II with boron containing reducing agent is effectively broken with the use of malic acid. This reduces the time and the process is easily scalable at commercial scale. Further, the inventors have developed a one-pot process for the preparation of azithromycin. The process does not involve the isolation of any intermediates, thereby reducing the work-up time as well as the cost of production. By following the present process, the yield of the final product, azithromycin, is also considerably improved.

The inventors have also developed a process for the preparation of stable form of anhydrous azithromycin by removing of moisture from azithromycin hydrates and crystallizing the anhydrous azithromycin from a suitable organic solvent. The inventors have also developed pharmaceutical compositions that contain the stable form of anhydrous azithromycin in admixture with one or more solid or liquid pharmaceutical diluents, carriers, and/or excipients.

A first aspect of the present invention provides a process for the preparation of azithromycin of Formula I wherein the process includes the steps of:
a) reducing 6,9 imino ether compound of Formula II with a boron containing reducing agent, to get a compound of Formula III or its boron complex;
b) optionally, breaking the boron complex to get the compound of Formula III; and
c) treating the compound of Formula III with formic acid and formaldehyde to get the azithromycin.

A second aspect of the present invention provides a process for the preparation of azithromycin wherein the process includes the steps of:
a) reducing 6,9 imino ether compound of Formula II with a boron containing reducing agent, to get a boron complex of compound of Formula III;
b) extracting the boron complex in one or more organic solvents;
c) treating the organic solvent containing the boron complex with malic acid;
d) N-methylating with formic acid and formaldehyde; and
e) isolating the azithromycin.

In general, the reduction of the compound of Formula II is carried out in the presence of water. Alternatively, it may be carried out in the presence of water containing one or more organic solvents. The organic solvents may include one or more of alcohols, haloalkanes, esters, ethers or aromatic solvents. The pH of the reaction mass can be maintained at 8 or less by the addition of a suitable acid or a buffering agent which include, but not limited to, formic acid, acetic acid, or the like. The boron containing reducing agents are known in the art and include, but not limited to, sodium borohydride, diborane, borane, trialkylboranes, dialkylalkoxyboranes, Vitride®, Lithium aluminium hydride, and the like.

The product of Formula III obtained is generally present in the form of its boron complex. In general, the boron complex so obtained can be converted to its free form by adjusting the pH of the mass to acidic range and then basifying, whereby the product is extracted from the mass using a suitable organic solvent. Alternatively, it can be extracted in a suitable organic solvent under basic conditions. The solvent may be concentrated and treated with malic acid in the presence of water. The pH can be brought down to about 2.5 with hydrochloric acid and the compound of formula III is extracted in an organic solvent. The resultant mixture may be treated with formic acid and formaldehyde mixture to give azithromycin. The azithromycin may be isolated and crystallized in acetonitrile-water/isopropyl alcohol-water mixture, which may be converted to its suitable hydrated or anhydrous form.

Suitable organic solvents include alcohols, haloalkanes, esters, ethers, aromatic solvents, acetonitrile, or mixtures thereof. Suitable alcohols include methanol, ethanol, n-propanol, isopropanol and butanol. The halogenated solvent includes halo (C₁-C₆) alkanes such as methylene chloride, chloroform, carbon tetrachloride, 1,1,1-trichloroethylene, 1,1,2-trichloroethylene, and the like. Examples of esters include ethyl formate, methyl acetate, ethylacetate, n-propyl acetate, isopropylacetate, isobutyl acetate, butyl acetate and amyl acetate. The aromatic solvents may include one or more of benzene, toluene, xylene, and n-hexane. The ethers may include one or more of diethyl ether, diisopropyl ether, tetrahydrofuran, and 1,4-dioxane. Mixtures of all of these solvents are also contemplated.

A third aspect of the present invention provides a process for the preparation of azithromycin wherein the process includes the steps of:
a) reducing 6,9 imino ether compound of Formula II with a boron containing reducing agent, to get a boron complex of compound of Formula III;
b) extracting the boron complex in one or more organic solvents;
c) N-methylating with formic acid and formaldehyde to get azithromycin boron complex;
d) isolating the azithromycin boron complex from reaction mass; and
e) breaking the boron complex of azithromycin to get the azithromycin.

The conversion of Formula II to azithromycin may be carried out in one-pot without isolation of intermediates. In particular, no intermediate is isolated from step a) to step c).

A fourth aspect of the present invention provides a process for the preparation of anhydrous azithromycin. The process includes the steps of:

a) removing the moisture from hydrated forms of azithromycin;
b) adding one or more water miscible solvents; and
c) isolating the anhydrous form of azithromycin by the removal of the solvents.

The solvent may be removed by a technique which includes, for example, distillation, distillation under vacuum, evaporation, filtration, filtration under vacuum, decantation and centrifugation.

Azithromycin used as starting material can be prepared by any method known in the art or by any one of the methods provided above. A solution of azithromycin obtained from the last stages of synthetic process can also be employed as the starting material. The so obtained azithromycin is subjected to removal of moisture present as hydrate. The moisture can be removed by drying under vacuum, dissolving or suspending azithromycin in a solvent capable of removing moisture by azeotropic distillation or by passing the solution of azithromycin through a bed of activated molecular sieves or variant thereof wherein the activated bed removes moisture. For removing moisture by azeotropic distillation, methylene chloride can be employed.

The so obtained azithromycin, which has lowered moisture content, is then taken up in a suitable water miscible organic solvent. The water miscible organic solvents include one or more of acetonitrile, ethanol, methanol, or the like. If desired, excess of such organic solvent can be used to make a clear solution of azithromycin in such solvent. After forming the solution, the solvent can be recovered to get a desired solubility profile of azithromycin.

In one aspect, the solution may be cooled before filtration to obtain better yields.

The product obtained may be further or additionally dried to achieve the desired moisture values. For example, the product may be further or additionally dried in a tray drier, dried under vacuum and/or in a Fluid Bed Dryer.

The process may produce the anhydrous azithromycin having a purity of more than 99% when measured by HPLC and a moisture content of about 1.0% w/w or less. In particular, it may produce the anhydrous azithromycin having a purity more than 99.5% by HPLC and a moisture content of about 0.7% or less.

The stability study performed on anhydrous form of azithromycin suggests that it is stable for more than 18 months under normal stability studies & for more than 3 months for accelerated stability studies.

It has been found that the anhydrous form of azithromycin is stable and there is no change in the related substance content of anhydrous form stored at 25±2° C. at 60±5% relative humidity & 40±2° C. at 75±5% relative humidity

It has been also found that after 12 months of storage at 25±2° C. at 60±5% relative humidity, anhydrous azithromycin was having a purity more than 99% by HPLC and a moisture content of about 0.6% or less.

It has been also found that after 3 months of storage at 40±2° C. at 75±5% relative humidity, anhydrous azithromycin was having a purity more than 98% by HPLC and a moisture content of about 0.6% or less.

While the present invention has been described in terms of its specific embodiments, certain modifications and equivalents will be apparent to those skilled in the art and are intended to be included within the scope of the present invention.

### EXAMPLE 1

### Preparation of Cyclic Amine (Formula III)

6,9-Imino ether (Formula-II) (100 g) was suspended in water (1.0 lit). The reaction mixture was cooled to 0-5° C. To the resulting reaction mixture, chilled aqueous solution of sodium borohydride (18.0 g in 200 ml water) was added at a temperature between 0-5° C. while maintaining the pH between 6.0-8.0 with formic acid. After completion of the borohydride addition, the reaction mixture was stirred at 0-5° C. for 1 hour and then at room temperature for 10 hours. It was then extracted with chloroform at pH 9.5. The chloroform layer was concentrated under vacuum and the residue so obtained was treated with water (1.0 lit) and malic acid (75 g). The pH of the resultant mixture was further adjusted to 2.5 with hydrochloric acid. The product was extracted in chloroform (500 ml×2) after adjusting the pH to about 9.5 and 10.0 with sodium hydroxide solution, whereby the compound of Formula III separated out from the reaction mass, which was isolated and dried.

Yield: 78.0 g.

### EXAMPLE 2

### Preparation of Anhydrous Azithromycin

(a) 6,9-Imino ether (Formula-II) (100 g) was suspended in water (1.0 lit). The reaction mixture was cooled to 0-5° C. To the resulting reaction mixture, chilled aqueous solution of sodium borohydride (18.0 g in 200 ml water) was added at temperature between 0-5° C. while maintaining the pH between 6.0-8.0 with formic acid. After completion of borohydride addition, the reaction mixture was stirred at 0-5° C. for 1 hr and then at room temperature for 10 hrs. After completion, the reaction mass is extracted with chloroform at pH 9.5. Chloroform layer was concentrated under vacuum and the residue was treated with water (1.0 lit) and malic acid (75 g). The pH of resultant mixture further adjusted to 2.5 with hydrochloric acid. The product was extracted in chloroform (500 ml×2) after adjusting the pH 9.5 to 10.0 with sodium hydroxide solution. The chloroform extracts were combined and taken for next step.

(b) The pH of the chloroform extract was adjusted to 5.0 to 5.5 with formic acid (17.0 g) and formaldehyde (17.0 g) and the resultant mixture was refluxed for 10 hr. After completion of the reaction, water (500 ml) was added and pH brought to 4.0 with hydrochloric acid. The pH of the reaction mixture was adjusted to 8.0 using sodium hydroxide solution and the aqueous layer was extracted with chloroform. The chloroform extract was concentrated to dryness. The residue obtained was dissolved in methylene chloride (750.0 ml), filtered and concentrated and dried under reduced pressure. The solid so obtained was dissolved in acetonitrile (750.0 ml). The acetonitrile was partially distilled out (600.0 ml) under vacuum. The slurry obtained was cooled to 10-25° C. and filtered to get anhydrous azithromycin.

Yield: 45.0 g.

Purity: 99.0%.

### EXAMPLE 3

### Preparation of Azithromycin

### (a) Preparation of Boron Complex of Cyclic Amine of Formula III:

6,9-Imino ether (Formula-II) (100 g) was suspended in water (1.0 lit). The reaction mixture was cooled to 0-5° C. To the resulting reaction mixture, chilled aqueous solution of sodium borohydride (18.0 g in 200 ml water) was added at temperature between 0-5° C. and pH was maintained between 6.0-8.0 with formic acid. After completion of the borohydride addition, the reaction mixture was stirred at 0-50° C. for 1 hr and then at room temperature for 10 hrs. The pH was adjusted to 9.5 with sodium hydroxide solution and then aqueous layer was extracted with chloroform (300 ml×2). The combined chloroform extracts containing title compound a) was taken for the next step.

(b) The pH of the chloroform extract was adjusted to 5.0 to 5.5 with formic acid (17.0 g) and formaldehyde (17.0 g) and the resultant mass was refluxed for 10 hr. After completion of the reaction, water (500 ml) was added and the pH was brought to 4.0 with hydrochloric acid. The chloroform layer was separated and to the aqueous layer, methanol (500 ml) was added. The resulting reaction mixture was cooled to −10 to −20° C. and the pH was adjusted to 1.0 with hydrochloric acid. Then reaction mixture was basified with sodium hydroxide and extracted with chloroform (300 ml×2). The combined chloroform extracts were concentrated to dryness and crystallized with isopropyl alcohol/water or acetonitrile/water mixture to get the title compound.

Yield: 65 g.

Purity: 98.0% (By HPLC)

### EXAMPLE 4

### Preparation of Azithromycin

The Cyclic amine of formula III obtained from example 1, was dissolved in chloroform (1 Litre) and adjusted to pH 5.0 to 5.5 with methylating mixture i.e. formic acid (17.0 g) and formaldehyde (17.0 g) and refluxed for 10 hr. The resultant mass was subjected to process as exemplified in example 3 (b), to get the title compound.

### EXAMPLE 5

### Anhydrous Azithromycin

Azithromycin (500 gm, moisture content=2.64%) was dissolved in dichloromethane (8.0 Lit). The solution was heated and the solvent was completely removed under vacuum. Acetonitrile (2.5 Lit) was added to the residue so obtained and the reaction mass was gradually cooled to 15 to 20° C. The resulting slurry was stirred at 15 to 20° C. for three hours and filtered; the solids so obtained were dried under, vacuum to get anhydrous azithromycin.

### Yield: 325 gm

Purity: 99.62% (by HPLC)

Moisture Content: 0.6% w/w.

While the present invention has been described in terms of its specific embodiments, certain modifications and equivalents will be apparent to those skilled in the art and are intended to be included within the scope of the present invention. For example, it is understood that the anhydrous azithromycin can be incorporated in dosage forms for treating conditions for which azithromycin is useful.

1. A process for the preparation of azithromycin, the process comprising:
a) reducing 6,9 imino ether compound of Formula II 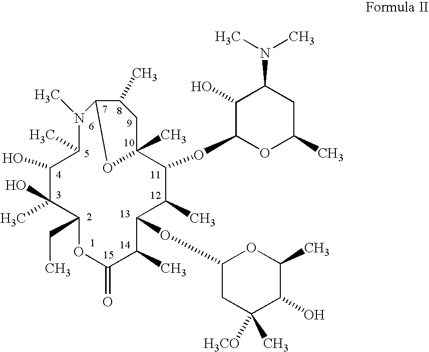
with a boron containing reducing agent in water, to get a compound of Formula III or its boron complex 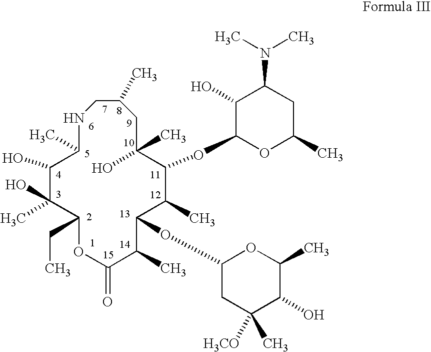
b) optionally, breaking the boron complex to get the compound of Formula III; and
c) treating the compound of Formula III or its boron complex with formic acid and formaldehyde to get the azithromycin.

2. The process of claim 1, wherein pH at step a) is maintained between 6.0 and 8.0 by addition of an acid or a buffering agent.

3. The process of claim 1, wherein the boron reducing agent comprises one or more of sodium borohydride, diborane, borane, trialkylboranes, dialkylalkoxyboranes, Vitride®, and lithium aluminium hydride.

4. The process of claim 1, wherein the boron complex is broken by one or both of acidification and basification.

5. (canceled)

6. (canceled)

7. A process for the preparation of azithromycin, the process comprising:
a) reducing 6,9 imino ether compound of Formula II 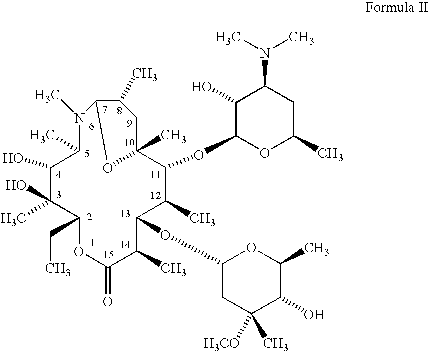
with a boron containing reducing agent in water, to get a boron complex of compound of Formula III 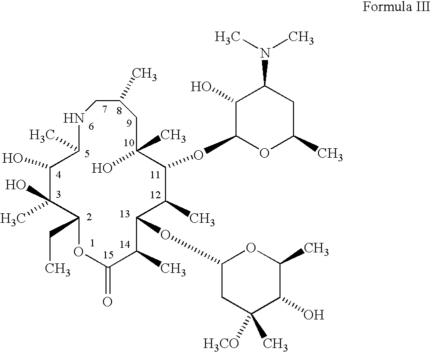
b) extracting the boron complex in one or more organic solvents;
c) N-methylating with formic acid and formaldehyde to get azithromycin boron complex;
d) isolating the azithromycin boron complex from reaction mass;
e) breaking the boron complex of azithromycin to get the azithromycin, and
h) isolating the azithromycin.

8. (canceled)

9. The process of claim 7, wherein the boron complex of the compound of Formula III is broken at acidic pH below 1.0 in the presence of alcoholic solvent

10. The process of claim 9, wherein the alcoholic solvent comprises one or more of methanol, ethanol, n-propanol, isopropanol and butanol.

11. A process for the preparation of anhydrous azithromycin, the process comprising:
a) removing the moisture from hydrated forms of azithromycin;
b) adding one or more water miscible solvents; and
c) isolating the anhydrous form of azithromycin by the removal of the solvents.

12. The process of claim 11, wherein a solution or a suspension of the hydrated forms of azithromycin is obtained in a solvent before removing the moisture.

13. The process of claim 12, wherein the moisture is removed by one or both of azeotropic distillation and passing through a bed of activated molecular sieves.

14. (canceled)

15. The process of claim 11, wherein the moisture is removed by drying under vacuum.

16. The process of claim 11, wherein the solvent comprises one or more of methylene chloride, chloroform, carbon tetrachloride, 1,1,1-trichloroethylene, 1,1,2-trichloroethylene, ethyl acetate toluene, diethyl ether, methyl acetate, n-propyl acetate, isopropyl acetate, isobutyl acetate, butyl acetate, methanol, ethanol, n-propanol, isopropanol butanol and acetonitrile.

17. The process of claim 11, wherein removing the solvent comprises one or more of distillation, distillation under vacuum, evaporation, filtration, filtration under vacuum, decantation, and centrifugation.

18. The process of claim 11, further comprising cooling prior to isolating the anhydrous azithromycin.

19. Anhydrous azithromycin having purity 99% or more when measured by HPLC.

20. The anhydrous azithromycin of claim 19 having purity greater than 99.5%.

21. Anhydrous azithromycin having moisture content of about 1.0% W/w or less.

22. The anhydrous azithromycin of claim 21 having moisture content of about 0.7% w/w or less.

23. (canceled)

24. (canceled)

25. Storage stable anhydrous form of azithromycin, wherein the azithromycin retains at least about 99% of its initial purity after one year, when stored at 25±2° C. at 60±5% relative humidity.

26. The storage stable anhydrous from of azithromycin of claim 25, wherein there is no change in related substances of the azithromycin.

27. Storage stable anhydrous form of azithromycin, wherein the azithromycin retains at least about 98% of its initial purity after three months, when stored at 40±2° C. at 75±5% relative humidity.

28. The storage stable anhydrous from of azithromycin of claim 27, wherein there is no change in related substances of the azithromycin.

29. A pharmaceutical composition comprising a therapeutically effective amount of a stable anhydrous azithromycin having purity more than 99% by HPLC; and one or more pharmaceutically acceptable carriers, excipients or diluents.

30. The process of claim 2, wherein the acid comprises one or more of formic acid, acetic acid or hydrochloric acid.

31. The process of claim 1, wherein steps (b) and (c) are carried out in one or more halogenated solvents.

32. The process of claim 31, wherein the halogenated solvent comprises one or more of methylene chloride, chloroform, carbon tetrachloride, 1,1,1-trichloroethylene and 1,1,2-trichloroethylene.

33. The process of claim 4, wherein the boron complex is broken in the presence of malic acid.

34. The process of claim 7, wherein the organic solvent comprises one or more of methylene chloride, chloroform, carbon tetrachloride, 1,1,1-trichloroethylene, 1,1,2-trichloroethylene, ethyl acetate, toluene and diethyl ether.

35. The process of claim 7 wherein the azithromycin is isolated at a basic pH.
